(19) United States
(12) Patent Application Publication
(10) Pub. No.: US 2005/0283218 A1
(43) Pub. Date: Dec. 22, 2005

(54) Implantable chamber for biological induction or enhancement of muscle contraction

(76) Inventor: Michael S. Williams, Santa Rosa, CA (US)

Correspondence Address: DEANNA J. SHIRLEY, 3418 BALDWIN WAY, SANTA ROSA, CA, 95403, US

(21) Appl. No.: 11/150,374

(22) Filed: Jun. 11, 2005

Related U.S. Application Data
(60) Provisional application No. 60/582,184, filed on Jun. 22, 2004.

(51) Int. Cl. A61N 1/05; A61F 2/02

(52) U.S. Cl. 607/119; 623/11.11

(57) ABSTRACT

A percutaneously implantable chamber for the treatment of a cardiac condition is disclosed herein, the chamber capable of delivery and maintenance of viable cells comprising a pacemaker gene or other genes intended to impart a specific function via a host cell. An artificial sinoatrial node and artificial atrial ventricular node for the restoration of the pacemaker function of the heart of a subject comprises a chamber comprising cells expressing a pacemaker gene. Further, a chamber may be used for the implantation and maintenance of viable, responsive, immunoisolated cells to induce or enhance muscle contraction of a subject for the treatment of a disorder.

### RELATED APPLICATIONS

This application is related to and claims the benefit of the priority date of Provisional U.S. patent application Ser. No. 60/582,184 titled “Implantable Chamber for Biological Induction or Enhancement of Muscle Contraction”, filed Jun. 22, 2004, by Williams.

### FIELD OF THE INVENTION

The invention herein is related to implantable medical devices and more specifically to devices and methods for inducing, restoring or enhancing muscle contraction. In a specific example, the invention is an artificial sinoatrial node or atrioventricular node of the mammalian heart. Further, the invention relates to a percutaneously implantable chamber for delivery and maintenance of viable cells and for the conduction of the pacemaker current from the cells within the chamber to the endogenous cardiac myocytes of a subject.

### BACKGROUND OF THE INVENTION

Specialized cardiac conducting tissue and the myocardium maintain an intrinsic rhythm in the healthy mammalian heart. The heart's rate is mediated through the autonomic nervous system which operates on a small mass of muscle cells called the sinoatrial (SA) node, which is located on the right atrium of the heart. An electrical signal generated by this structure causes the atria of the heart to contract. Contraction of the atria forces blood into the ventricles of the heart. The signal from the SA node is propagated to the ventricles through a structure called the atrioventricular (AV) node after a brief delay. The signal from the AV node causes the ventricles to contract, forcing the blood throughout the body.

Many forms of heart disease impair the function of the SA and AV nodes and their associated conductive tissues, and can lead to abnormalities of the heart rhythm. These abnormalities, generally referred to as arrhythmias, potentially lead to substantial patient discomfort or even death. Morbidity and mortality from such problems is significant to the public health. In the United States alone for example, cardiac arrest accounts for 220,000 deaths per year, possibly more than 10% of total American deaths.

Implantable medical devices developed for the management of cardiac rhythm, referred to herein as pacemakers, have been helpful and even life saving for a substantial number of patients suffering cardiac arrhythmia A typical pacemaker includes a pulse generator, a power source, a pacing lead, electronic circuitry, and a programmer. The pulse generator sends electrical stimulation pulses through the pacing leads to stimulate the heart to beat in a controlled rhythm. Advanced pacemakers may include physiological sensors in order to provide pacing that is responsive to a patient's level of activity and other varying physiological demands. However, such devices are unable to perform the complex physiological functions of normal, healthy cardiac cells. Additionally, such advances require additional circuitry and increase the demands of the power source, thereby competing with the desire for smaller, affordable and longer lasting devices. Drawbacks of all pacemakers include the need for maintenance and power source replacement.

It is therefore desirable to provide a device and method for increasing and/or restoring the physiological function of the natural cardiac pacemaker cells and the myocardium. In addition to being maintenance free, such cells will be naturally responsive to emotional and hormonal changes and varied activity levels of a patient, and are a curative solution to the disease state, rather than a palliative measure.

Some advances have been made in the development of biological cell lines that record a pacemaker current and consequently, in theory, are able to perform the cardiac pacemaker function. Such advances also hold some promise for advances in the treatment of other disorders related to muscle contraction, including, for example, stress incontinence. Further, the technology may be used in targeted muscle contraction to regulate food intake for the treatment of obesity. However, there remains a need in the art for a device and a method by which to deliver such cells to the desired site of functioning in a minimally invasive manner. Further, there remains a need for preventing the migration of cells from the desired site following delivery. If the cells are contained in order to prevent migration, the containment device must be suitable for the continued viability of cells. For example, the device must permit the entry and exit of materials necessary for and resulting from cellular respiration, such as, for example, oxygen, nutrients, electrolytes, carbon dioxide, and lactic acid. While allowing the entry and release of desired materials, the containment device must not permit the entry of antibodies where non-autologous cells are utilized. It is also desirable that the device itself not provoke an excessive immune response.

Still further, the containment must not prohibit the formation of cell-cell gap junctions between the implanted cells and the endogenous cells. The device must permit the electrical conductivity of the pacemaker current generated by the cells to the endogenous cardiac myocytes. The device's surfaces must be non-fouling, and prevent encapsulation by overgrowth of cells, or, in the alternative, promote endogenous cell growth and neovascularization.

### SUMMARY OF THE INVENTION

The invention herein relates to an implantable chamber for the delivery and maintenance of viable cells for the treatment of a cardiac condition. The cardiac condition may be a cardiac rhythm disorder. The implantable chamber for the delivery and maintenance of cells for the treatment of a cardiac condition may comprise one or more walls defining a substantially hollow interior. The one or more walls comprise one or more pores, said one or more pores configured to allow the passage of molecules related to the cells' respiration, such as for example, oxygen, nitrogen, nutrients, carbon dioxide, and lactic acid. The pores are also large enough to permit a neurohormonal interface and exchange between the cells and the blood. The pores are, however, configured to prevent the passage of the cells therefrom. The pores may be generally between 0.1 micrometer and 10 micrometers in diameter. The membrane is also configured to allow electrical conductivity from the interior of the chamber to the exterior of the chamber, whether the current results from the formation of cell-cell gap junctions between the cells or from a depolarization of the cells, or other mechanism. The pores are further configured to prevent the passage of antibodies or endogenous cells therethrough. The device may comprise metals or polymers or both, and may comprise a plurality of sintered spherical structures surrounding the cells, the sintered structure further encapsulated by a polymer membrane, through which electrical current is conducted to the endogenous cells. Examples of suitable metals include stainless steel, nickel titanium, and suitable polymers include, for example, ePTFE, or any membrane prepared using a suitable nanopore membrane technology.

The chamber may further comprise one or more anchors which may be formed from a shape memory metal or polymer. It may comprise a delivery configuration and a deployed configuration, enabling percutaneous implantation of the device. The cells housed in the chamber may comprise a gene that expresses any one or more of numerous proteins or subunits that play a role in regulating heartbeat. The cells are capable of recording a current by forming cell-cell gap junctions, through a depolarization phase or other mechanism and are capable of conducting an electrical current to the endogenous cardiac cells.

The chamber may further comprise one or more conductive wires extending therefrom and/or an electrically conductive grid. Such wire or wires and grid may enhance conductivity from the cell culture to the enodogenous cardiac myocytes. The grid, like the chamber itself, may be treated to prevent the overgrowth of endogenous cells, or alternatively, treated to enhance the overgrowth of endogenous cells or to promote neovascularization.

The chamber may house stem cells treated to express a pacemaker gene by electroporation, transfer through liposomes, a plasmid, a viral vector, dendrimers, cationic polymers, nanohydrogels, crosslinked micelles, cell-penetrating peptides, cell targeting peptides or other suitable method including both viral and non-viral vectors. When said chamber is implanted in a subject, it allows conductivity of electrical impulses from the cells within the chamber to the endogenous cardiac myocytes of the subject. The chamber may be percutaneously implantable in the atrial septal wall or the ventricular septal wall of a subject.

An artificial sinoatrial node is disclosed, comprising an implantable chamber comprising viable cells comprising a pacemaker gene. An artificial atrioventricular node is also disclosed, comprising an implantable chamber comprising viable cells comprising a pacemaker gene.

A method according to the invention for the minimally invasive treatment of a cardiac condition, including a cardiac rhythm disorder, may comprise the steps of: providing a chamber comprising viable cells, said chamber comprising a delivery configuration and a deployed configuration; accessing the right atrium of a subject; creating an aperture in the atrial septal wall; delivering the chamber to the aperture in the atrial septal wall; and deploying the chamber within the aperture in the atrial septal wall. The chamber may comprise one or more anchors, with the added step of deploying the one or more anchors for securing the chamber within the atrial septal wall. The cells may comprise a pacemaker gene. The step of accessing the right atrium may comprise the steps of accessing the femoral vein and the inferior vena cava with a catheter.

An alternative method according to the invention may comprise accessing the ventricular septal wall, creating an aperture therein, and delivering the chamber to the aperture and deploying it therein.

The chamber is configured to maintain said viable cells following delivery of said chamber. It is also configured to allow electrical conductivity from the interior of said chamber to the exterior of the chamber and the endogenous cells of a subject. It may be configured to comprise one or more walls comprising one or more pores, said one or more pores configured to allow the passage of molecules related to the cells' respiration, said one or more pores configured to prevent the passage of the cells therefrom.

A device according to the invention may be configured to be readily removed from the subject. Further, the chamber within the device may be exchanged from the device to be refilled or replaced with an alternate chamber.

An implantable chamber for implanting viable, immunoisolated, electrically conductive cells into a subject for treatment to induce or enhance muscle contraction is disclosed herein

### BRIEF DESCRIPTION OF THE DRAWINGS

FIG. 1 is a partial cutaway illustration of the human heart in which an embodiment according to the invention has been implanted.

FIG. 2A is a perspective view of an embodiment according to the invention in its deployed configuration.

FIG. 2B is an exploded view of the embodiment of FIG. 2A.

FIG. 3 is a plan view of an alternative embodiment according to the invention.

FIG. 4 is a side view of a membrane useful according to the invention.

FIG. 5 is an alternative membrane useful according to the invention.

FIG. 6 is a perspective view of an alternative embodiment according to the invention.

FIG. 7 is a perspective view of an alternative embodiment according to the invention.

FIG. 8 is a perspective view of an alternative embodiment according to the invention.

FIG. 9A illustrates a perspective view of an alternative embodiment according to the invention in its deployed configuration.

FIG. 9B illustrates an exploded view of the embodiment of FIG. 9A.

FIG. 9C illustrates a side view of the embodiment of FIG. 9A

### DETAILED DESCRIPTION OF THE INVENTION

A “self-expanding” device has the ability to revert readily from a reduced profile configuration to a larger profile configuration in the absence of a restraint upon the device that maintains the device in the reduced profile configuration.

“Expandable” refers to a device that comprises a reduced profile configuration and an expanded profile configuration.

“Expansion ratio” refers to the percentage increase in diameter of a device following conversion of the device from its reduced profile configuration to its expanded profile configuration.

“Elasticity” refers to the ability of a material to repeatedly undergo significant tensile stress and strain, and/or compression stress and strain, and return to its original configuration.

A “switching segment” comprises a transition temperature and is responsible for the shape memory polymer's ability to fix a temporary shape.

A “thermoplastic elastomer” is a shape memory polymer comprising crosslinks that are predominantly physical crosslinks.

A “thermoset” is a shape memory polymer comprising a large number of crosslinks that are covalent bonds.

Although a device according to the invention may be manufactured from a suitable metal, it may alternatively be manufactured from a polymer, such as, for example, expanded polytetrafluoroethylene (ePTFE) which may vary in porosity. A device comprising polymeric materials has the advantage of compatibility with magnetic resonance imaging, potentially a long term clinical benefit. Further, if the more conventional diagnostic tools employing fluoroscopic visualization continue as the technique of choice for delivery and monitoring, radiopacity can be readily conferred upon polymeric materials. The use of polymeric materials in the fabrication of devices confers the advantages of improved flexibility, compliance and conformability, enhancing percutaneous delivery.

Examples of conductive polymers include, but are not limited to: polyaniline, polythiophene and their derivatives, and others.

Although the invention herein is not limited as such, portions of some embodiments of the invention comprise materials that are bioerodible. “Erodible” refers to the ability of a material to maintain its structural integrity for a desired period of time, and thereafter gradually undergo any of numerous processes whereby the material substantially loses tensile strength and mass. Examples of such processes comprise hydrolysis, enzymatic and non-enzymatic degradation, oxidation, enzymatically-assisted oxidation, and others, thus including bioresorption, dissolution, and mechanical degradation upon interaction with a physiological environment into components that the patient's tissue can absorb, metabolize, respire, and/or excrete. Polymer chains are cleaved by hydrolysis and are eliminated from the body through the Krebs cycle, primarily as carbon dioxide and in urine. “Erodible” and “degradable” are intended to be used interchangeably herein.

“Embedded” agents are set upon and/or within a mass of material by any suitable means including, but not limited to, combining the agent with the material while the material (such as, for example, a polymer) is in solution, combining the agent with the material when the material is heated near or above its melting temperature, affixing the agent to the surface of the material, and others. Examples of methods of embedding agents utilizing a solvent in a supercritical state are set forth in U.S. patent application Ser. Nos. 10/662,757 and 10/662,621, and are incorporated as if fully set forth herein.

“Balloon expandable” refers to a device that comprises a reduced profile configuration and an expanded profile configuration, and undergoes a transition from the reduced configuration to the expanded configuration via the outward radial force of a balloon expanded by any suitable inflation medium.

The term “balloon assisted” refers to a self-expanding device the final deployment of which is facilitated by an expanded balloon.

As used herein, a device is “implanted” if it is placed within the body to remain for any length of time following the conclusion of the procedure to place the device within the body.

The term “diffusion coefficient” refers to the rate by which a substance elutes, or is released either passively or actively from a substrate.

Unless specified, suitable means of attachment may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric. A polymer is a shape memory polymer if the original shape of the polymer is substantially recovered by heating it above a shape recovering temperature (defined as the transition temperature of a soft segment) even if the original molded shape of the polymer is destroyed mechanically at a lower temperature than the shape recovering temperature, or if the memorized shape is recoverable by application of another stimulus. Such other stimulus may include but is not limited to pH, salinity, hydration, and others. Shape memory polymers are highly versatile, and many of the advantageous properties listed above are readily controlled and modified through a variety of techniques. Several macroscopic properties such as transition temperature and mechanical properties can be varied in a wide range by only small changes in their chemical structure and composition.

As used herein, the term “segment” refers to a block or sequence of polymer forming part of the shape memory polymer. The terms hard segment and soft segment are relative terms, relating to the transition temperature of the segments. Generally speaking, hard segments have a higher glass transition temperature than soft segments, but there are exceptions. Natural polymer segments or polymers include but are not limited to proteins such as casein, gelatin, gluten, zein, modified zein, serum albumin, and collagen, and polysaccharides such as alginate, chitin, celluloses, dextrans, pullulane, and polyhyaluronic acid; poly(3-hydroxyalkanoate)s, especially poly(.beta.-hydroxybutyrate), poly(3-hydroxyoctanoate) and poly(3-hydroxyfatty acids).

Representative natural erodible polymer segments or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Suitable synthetic polymer blocks include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, arboxymethyl cellulose, cellulose triacetate and cellulose sulfate sodium salt. These are collectively referred to herein as “celluloses”.

Examples of synthetic degradable polymer segments or polymers include polyhydroxy acids, polylactides, polyglycolides and copolymers thereof, poly(ethylene terephthalate), poly(hydroxybutyric acid), poly(hydroxyvaleric acid), poly[lactide-co-(epsilon-caprolactone)], poly[glycolide-co-(epsilon-caprolactone)], polycarbonates, poly-(epsilon caprolactone) poly(pseudo amino acids), poly(amino acids), poly(hydroxyalkanoate)s, polyanhydrides, polyortho esters, and blends and copolymers thereof.

Rapidly erodible polymers such as poly(lactide-co-glycolide)s, polyanhydrides, and polyorthoesters, which have carboxyl groups exposed on the external surface as the smooth surface of the polymer erodes, also can be used. In addition, polymers containing labile bonds, such as polyanhydrides and polyesters, are well known for their hydrolytic reactivity. Their hydrolytic degradation rates can generally be altered by simple changes in the polymer backbone and their sequence structure.

Examples of suitable hydrophilic polymers include but are not limited to poly(ethylene oxide), polyvinyl pyrrolidone, polyvinyl alcohol, poly(ethylene glycol), polyacrylamide poly(hydroxy alkyl methacrylates), poly(hydroxy ethyl methacrylate), hydrophilic polyurethanes, HYPAN, oriented HYPAN, poly(hydroxy ethyl acrylate), hydroxy ethyl cellulose, hydroxy propyl cellulose, methoxylated pectin gels, agar, starches, modified starches, alginates, hydroxy ethyl carbohydrates and mixtures and copolymers thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), and copolymers and blends thereof. Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

An additional advantage of polymers includes the ability to control and modify properties of the polymers through the use of a variety of techniques. According to the invention, optimal ratios of combined polymers, optimal configuration of polymers synthesized to exhibit predictable rates of erosion, and optimal processing have been found to achieve highly desired properties not typically found in polymers. In general, erosion of a polymer will progress at a known range of rates. Environmental factors such as pH, temperature, tissue or blood interaction and other factors such as structural design of the device all impact the degradation rate of erodible polymers. Depending upon the desired performance characteristics of a device, in some cases it may be desirable to either “program in” a desired rate of erosion, or desired cycle of varied rates of erosion, to initiate on-demand erosion of a device, or to have a set of desired mechanical properties or to function in a desired manner for a period of time, and an alternative set of desired mechanical properties for a second period of time. For example, it may be desirable for the device to deliver a therapeutic substance under particular conditions and/or during a particular time period.

According to the invention, a polymer may be tailored to erode rapidly during one phase, such as, for example, a therapy delivery phase, followed by a period of time during which the polymer erodes at a slower rate. Such a time period of slower erosion may be followed by a second drug delivery phase during which the polymer again erodes rapidly. Similarly, a polymer may be tailored to erode on demand, upon the introduction of a stimulus such as increase in temperature, exposure to radiation, and/or others. Any number of combinations of desired phases is possible according to the invention.

The rate of erosion of a polymer may be controlled by one or more of several techniques. An example of such a technique includes the incorporation of an agent or substance that acts as a catalyst of degradation upon exposure to a stimulus. Examples of such agents or substances include, but are not limited to, sensitizers, dissolution inhibitors, biochemically active additives, thermal, light, electromagnetic radiation, or enzyme-activated catalysts, or some combination of the foregoing. Examples of sensitizers include, but are not limited to photoacid generators (PAGs), dissolution inhibitors, and radiosensitizers. Examples of biochemically active additives include, but are not limited to, lipids. Further, one or more layers of polymer comprising one of the foregoing agents may alternate with a layer of polymer that does not comprise such an agent, or is tailored to erode at a different rate or upon the introduction of an alternate stimulus. More specific examples of the foregoing in set forth in provisional U.S. patent application Ser. No. 60/633,494, and are incorporated as if set forth fully herein.

According to another aspect of the invention, surface treatment including, but not limited to removal of impurities and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”). Compressing a gas normally causes a phase separation and the appearance of a separate liquid phase. However, all gases have a critical temperature above which the gas cannot be liquefied by increasing pressure, and a critical pressure or pressure which is necessary to liquefy the gas at the critical temperature. For example, carbon dioxide in its supercritical state exists as a form of matter in which its liquid and gaseous states are indistinguishable from one another. For carbon dioxide, the critical temperature is about 31 degrees C. (88 degrees D) and the critical pressure is about 73 atmospheres or about 1070 psi.

The term “supercritical carbon dioxide” as used herein refers to carbon dioxide at a temperature greater than about 31 degrees C. and a pressure greater than about 1070 psi. Liquid carbon dioxide may be obtained at temperatures of from about −15 degrees C. to about −55 degrees C. and pressures of from about 77 psi to about 335 psi. One or more solvents and blends thereof may optionally be included in the carbon dioxide. Illustrative solvents include, but are not limited to, tetrafluoroisopropanol, chloroform, tetrahydrofuran, cyclohexane, and methylene chloride. Such solvents are typically included in an amount, by weight, of up to about 20%.

In general, carbon dioxide may be used to effectively lower the glass transition temperature of a polymeric material to facilitate the infusion of pharmacological agent(s) into the polymeric material. Such agents include but are not limited to hydrophobic agents, hydrophilic agents and agents in particulate form. For example, following fabrication, a device and a hydrophobic pharmacological agent may be immersed in supercritical carbon dioxide. The supercritical carbon dioxide “plasticizes” the polymeric material, that is, it allows the polymeric material to soften at a lower temperature, and facilitates the infusion of the pharmacological agent into the polymeric device or polymeric coating of a stent at a temperature that is less likely to alter and/or damage the pharmacological agent.

As an additional example, a device and a hydrophilic pharmacological agent can be immersed in water with an overlying carbon dioxide “blanket”. The hydrophilic pharmacological agent enters solution in the water, and the carbon dioxide “plasticizes” the polymeric material, as described above, and thereby facilitates the infusion of the pharmacological agent into a polymeric device or a polymeric coating of a device.

As yet another example, carbon dioxide may be used to “tackify”, or render more fluent and adherent a polymeric device or a polymeric coating on a device to facilitate the application of a pharmacological agent thereto in a dry, micronized form A membrane-forming polymer, selected for its ability to allow the diffusion of the pharmacological agent therethrough, may then applied in a layer over the device. Following curing by suitable means, a membrane that permits diffusion of the pharmacological agent over a predetermined time period forms. Surface treatment for the removal of impurities or the incorporation of a therapeutic substance are more fully set forth in commonly owned U.S. patent application Ser. Nos. 10/662,621 and 10/662,757, which are hereby incorporated in their entirety as if set forth fully herein.

Objectives of therapeutic substances incorporated into materials forming or coating an device according to the invention include reducing the adhesion and aggregation of platelets at the site of arterial injury, block the expression of growth factors and their receptors; develop competitive antagonists of growth factors, interfere with the receptor signaling in the responsive cell, promote an inhibitor of smooth muscle proliferation. Anitplatelets, anticoagulants, antineoplastics, antifibrins, enzymes and enzyme inhibitors, antimitotics, antimetabolites, anti-inflammatories, antithrombins, antiproliferatives, antibiotics, anti-angiogenesis factors, and others may be suitable.

“Cells” most often are adult allograft mesenchymal stem cells, but may alternatively be embryonic stem cells or any cells suitable for the expression of one or more pacemaker genes. The cells have been encoded with a desirable gene according to any suitable method including, but not limited to, electroporation, transfer through liposomes, a plasmid, a viral vector, dendrimers, cationic polymers, nanohydrogels, crosslinked micelles, cell-penetrating peptides, cell targeting peptides or other suitable method. Said cells may be terminally differentiated and/or terminally quiescent. The cells may be autograft, allograft, xenograft, or some combination thereof.

“Pacemaker gene” may include any one of the genes that express one or more of the proteins or subunits that play a role in regulating heart rate, including, but not limited to, any of the family of hyperpolarization activated cyclic nucleotide gated (HCN) ion channels, minimal potassium channel proteins or minimal potassium channel related peptides. Expression of pacemaker genes in stem cells has been reported and pacemaker current recorded from such cells in, for example, U.S. Patent Application Publication No. 2002/0187948, which is incorporated by reference herein in its entirety.

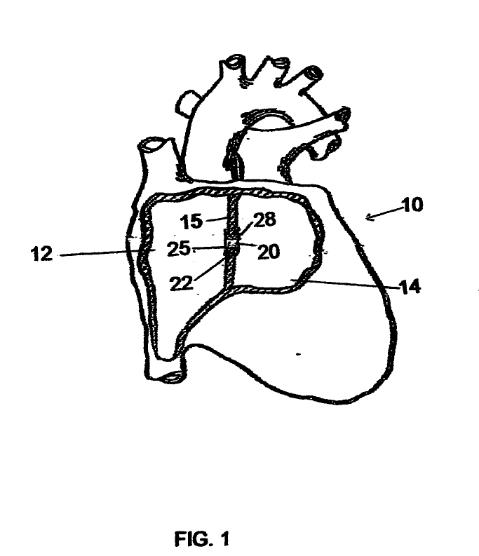

FIG. 1 illustrates a frontal view of human heart 10, with a partial cutaway to expose right atrium 12, left atrium 14 and interatrial septum 15. Biological pacemaker 20 is shown, deployed at interatrial septum 15. Biological pacemaker 20 is deployed through septal orifice 22, an artificial foramen ovale created in order to accommodate biological pacemaker 20. Chamber 25 houses cells 27 (not shown), most often in immunoisolation. Anchors 28, which may comprise, for example, stainless steel, or a shape memory material such as nickel titanium or a polymer, clamp to opposite sides of interatrial septum 15 and hold biological pacemaker 20 in place. Biological pacemaker 20 may be delivered percutaneously in a catheter in its delivery configuration (not shown), via, for example, an incision to access the femoral vein, to the inferior vena cava and ultimately the right atrium and septal wall therein. Biological pacemaker 20 may alternatively be delivered to the ventricular septal wall. After tracking biological pacemaker 20 into the right atrium, it is tracked further through the tricuspid valve and into the right ventricle. An aperture is then placed in the ventricular septal wall, and biological pacemaker 20 deployed therein.

The cells (not shown) housed in chamber 25 have been prepared via suitable means, such as, for example, electroporation, to express a pacemaker gene. Following preparation in, for example, a bioreactor, and being cultured to a sufficient population, the cells are loaded into chamber 25, which is then sealed.

Cell growth and expression of a pacemaker gene occurs within cell chamber 25, which prevents migration of the cells. Electrical current is conducted from the isolated cells within chamber 25, to the endogenous cardiac myocytes and throughout the heart in order to augment or restore lost pacemaker function of the heart, first in proximity to the natural SA node.

The membrane of chamber 25 is specially designed to comprise pores (not shown) of sufficient size to allow nutrient and metabolite transfer between the cells and the blood. Such nutrients and metabolites include, for example, oxygen, nitrogen, carbon dioxide, and lactic acid. The cells are exposed to oxygenated blood of the left atrium. The pores also permit a neurohormonal interface and exchange between the implanted cells and the blood of the subject. The pores however are too small to allow either cell migration or escape or to permit the entrance of cells or antibodies. Such pores are generally between approximately 0.1 micrometer and 10 micrometers in diameter, and sized to allow passage of molecules of a molecular weight of approximately 100,000 or less.

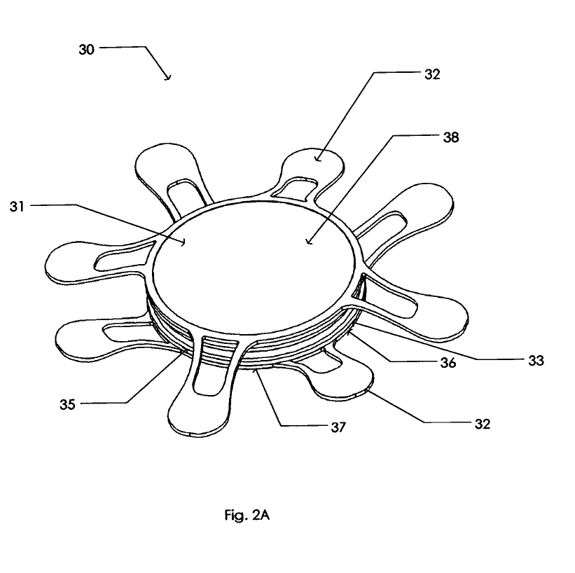

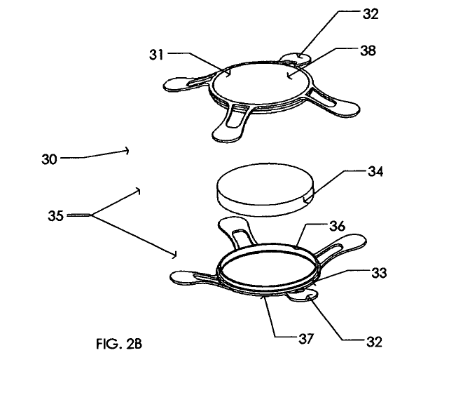

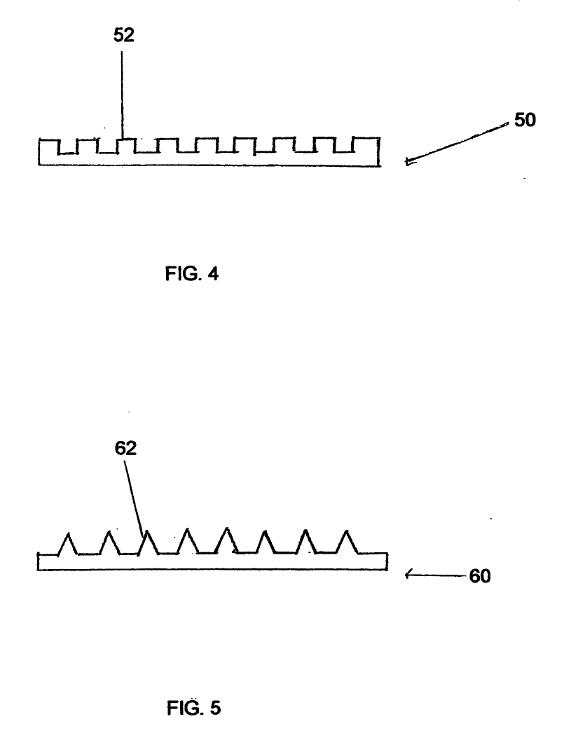

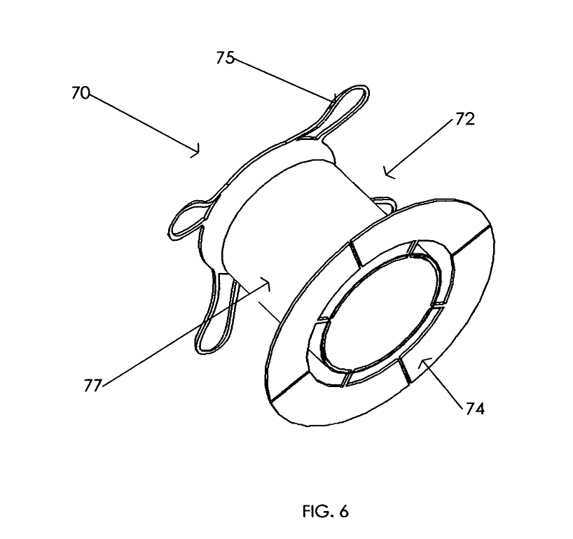

The membrane also allows the cells in the interior of chamber 25 to conduct electrical current from the interior of chamber 25 to the endogenous cells of a subject, through the formation of cell-cell gap junction formation, phase change or other suitable mechanism. The membrane of chamber 25 is generally less than or equal to approximately 500 micrometers in thickness. The structure of the surface of the membrane may be varied to allow for strength and increased surface area for increased oxygen contact and increased electrical conductivity, examples of which are further illustrated in FIGS. 4 and 5 below. Further, portions of the membrane may comprise varied porosity in order to maximize the function of the particular portion of membrane. Portions of the membrane most exposed to blood interface may be, for example, designed to maximize nutrient transfer, while portions of the membrane disposed to conduct current may be designed to maximize conduction from the interior of chamber 25 to the exterior of chamber 25. (See FIG. 2A et seq.)

Biological pacemaker 20 and anchors 28 may be reversibly deployable, allowing release of the device from the atrial septal wall or the ventricular septal wall and retrieval via catheter. Accordingly, biological pacemaker 20 may be removed from a subject. Further, chamber 25 may be exchangeable from biological pacemaker 20, allowing replacement of the cells within chamber 25 or replacement of chamber 25 with an alternative chamber.

FIGS. 2A and 2B illustrate in more detail from a perspective view from first end 31 of an embodiment according to the invention in its deployed configuration Biological pacemaker 30 comprises anchors 32 disposed at both first end 31 and second end 37 for securing biological pacemaker 30 on opposite sides of a septal wall of a subject. Biological pacemaker 30 also comprises chamber 35, which houses a population of cells 34 for the expression of a pacemaker gene and conduction of electrical current from the cells in the chamber to the cardiac myocytes of the patient. Sides 33 of chamber 35 comprise porous membrane 36, and first end 31 and second end 37 comprise porous membrane 38. Porous membranes 36 and 38 comprise pores having functions similar to those described in relation to FIG. 1. When biological pacemaker 30 is implanted in a patient, first end 31 and second end 37 interface most with the blood of the right and left atria respectively (see FIG. 1). Accordingly, membrane 38 may comprise a different porosity than membrane 36, in order to, for example, maximize entry of oxygen or achieve another objective of chamber 35 related to interface with the blood of a subject. Similarly, the porosity of membrane 36 of side 33 may be specifically designed to, for example, maximize the conduction of electrical current from the interior of chamber 35 to the septal wall of the subject.

Similar to the embodiment discussed in relation to FIG. 1 above, biological pacemaker 30 and anchors 32 may be reversibly deployable, allowing removal of biological pacemaker 30 from a subject in a minimally invasive manner. Further, chamber 35 is removable from biological pacemaker 30, allowing refilling or replacement of chamber 35.

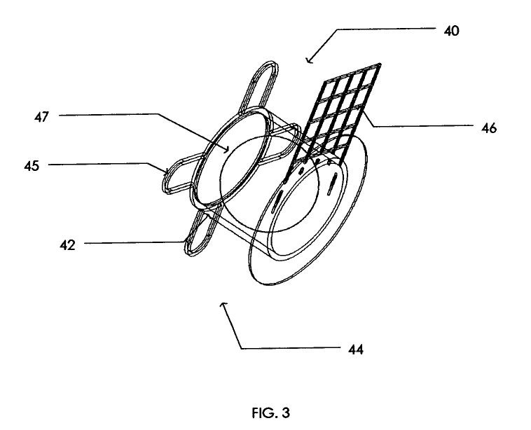

FIG. 3 illustrates an alternative embodiment according to the invention. Biological pacemaker 40 comprises chamber 42. Within chamber 42 synthetic conductive interface 43, conducting electrode 44 and grid 46. Synthetic conductive interface 43 comprises a hollow spherical structure formed of many smaller conductive spherical structures sintered together. Within chamber 42 is a population of pacemaker cells of suitable size to record a pacemaker current. When implanted in a subject, synthetic conductive interface 43, in electrical contact with the cells and with electrode 44 and grid 46, conducts the electrical current from the cells to these structures. Electrode 44 and grid 46 further conduct electrical current to a relatively large surface area of the endogenous cardiac myocytes of a patient. Biological pacemaker 40 may alternatively or additionally comprise one or more conductive fibers extending from the interior of chamber 42 and synthetic conductive interface 43 to the exterior of the device thus forming a conductive electrical conduit between internally isolated cells and endogenous cardiac myocytes. Grid 46 may comprise one of many structures and materials suitable for increased conductivity. Further, grid 46 may be treated to either prevent the overgrowth of endogenous cells, or, alternatively, to enhance the growth of the endogenous cells over grid 46.

Pacemaker 40 also comprises one or more anchors 45 that may comprise one or more shape memory materials and may be reversibly deployable. Further, or in the alternative, chamber 42 may be readily exchangeable with a replacement chamber (not shown) following implantation in a subject.

Membrane 47 of chamber 42 comprises pores (not shown) having similar characteristics to those described in relation to FIG. 1 in order to allow the entry and exit of molecules needed for and excreted by the cells during respiration, to allow the cells to be responsive to neurohormonal changes of the subject, and to prevent the migration of the cells and the entry of antibodies or endogenous cells. Outer membrane 47 of chamber 42 may further comprise a matrix construct comprising materials and structure designed for example, for increased oxygen exposure or enhanced electrical conductivity. Further, the membrane may comprise, for example, porous ePTFE, or a membrane prepared according to any suitable nanopore membrane technology, including, but not limited to, stereolithography or soft lithography. The outer membrane may further be treated to either prevent cell growth on the exterior of chamber 42, or, alternatively, to enhance cell growth and neovascularization, or otherwise comprise one or more therapeutic agents.

FIGS. 4 and 5 illustrate side views of alternate configurations of membrane suitable for use in the construction of a chamber according to the invention Membrane 50 of FIG. 4 comprises projections 52 for enhanced electrical conductivity, increased oxygen contact surface area and increased structural strength Similarly, membrane 60 of FIG. 5 comprises projections 62 for enhanced electrical conductivity and increased oxygen contact surface area Both cell viability and device performance are ensured when one of the foregoing membrane structures are utilized.

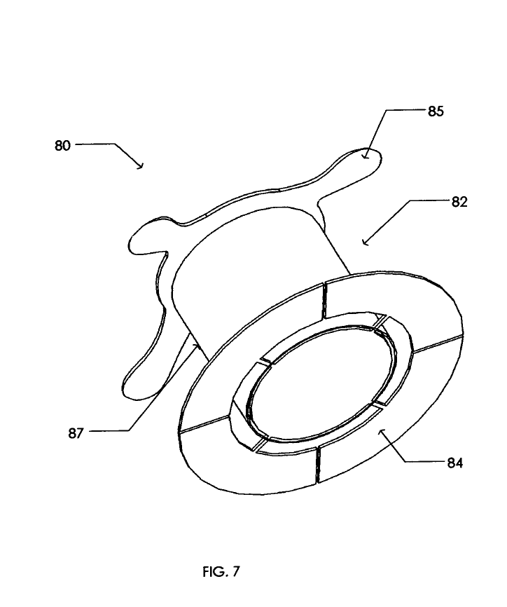

FIGS. 6 and 7 illustrate, from a perspective view of each, examples of variations on an alternative embodiment. Biological pacemaker 70 comprises chamber 72 comprising suitable membrane 77, one or more anchors 75. Biological pacemaker 70 further comprises electrode 74, comprising a relatively large surface area, for conducting electrical current from pacemaker cells (not shown) maintained within chamber 72 and to the endogenous cardiac myocytes of a subject. Comparable biological pacemaker 80, illustrated in FIG. 7, comprises anchors 85 of an alternate solid configuration. Further, electrode 84 comprises a discontinuous design, allowing conduction of current to a relatively large surface area with less material.

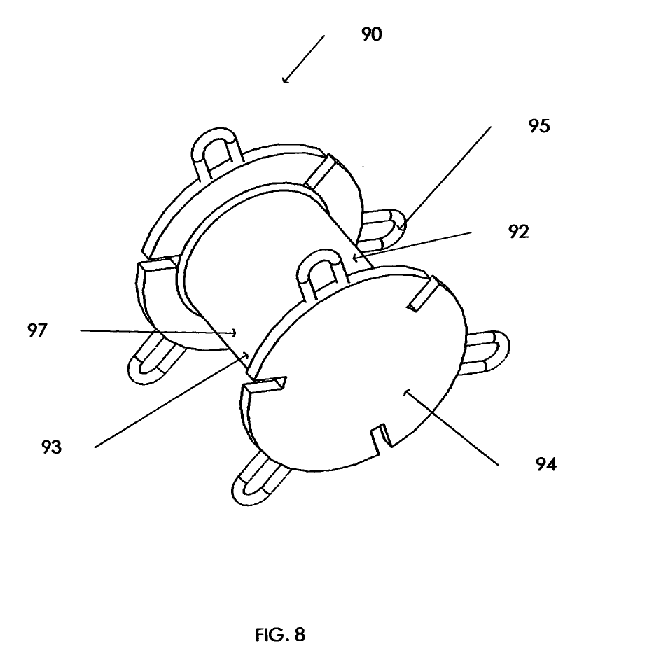

FIG. 8 is an example of yet another embodiment, showing the physical structure of a sample device design. Anchors 95 are unitary structures extending the length of biological pacemaker 90. In the interior of chamber 92 is synthetic conductive interface 93. Synthetic conductive interface comprises a plurality of small conductive spherical structures sintered together. Within synthetic conductive interface 93 is a cell population (not shown) of suitable size to record a pacemaker current. On the exterior of synthetic conductive interface 93 is porous membrane 97 comprising pores configured to allow the entry of substances needed for maintenance of the viable cells, to allow entry of neurohormonal substances to which the cells are responsive, to allow the exit of the cells' waste products, and to prevent the migration of the cells and the entry of antibodies or endogenous cells. Further, porous membrane 97 does not prevent electrical conductivity between the implanted cells and the endogenous cells of the subject. Electrode 94 further enhances the conduction of current from the implanted cells to the endogenous cells.

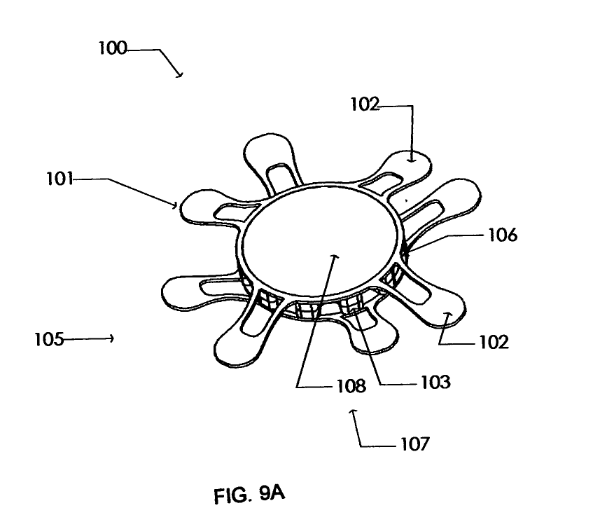

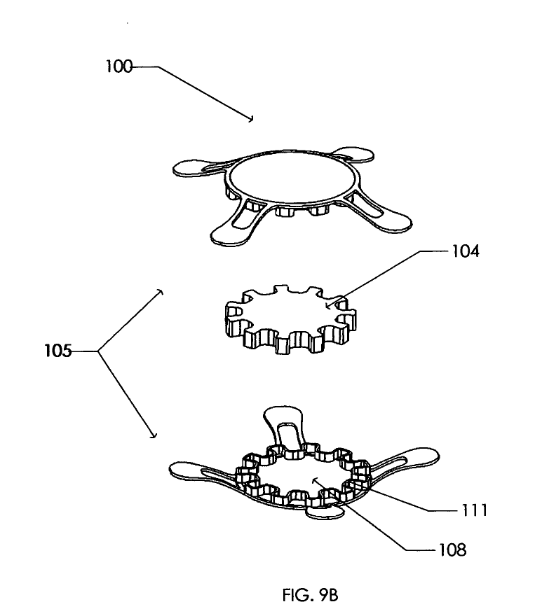

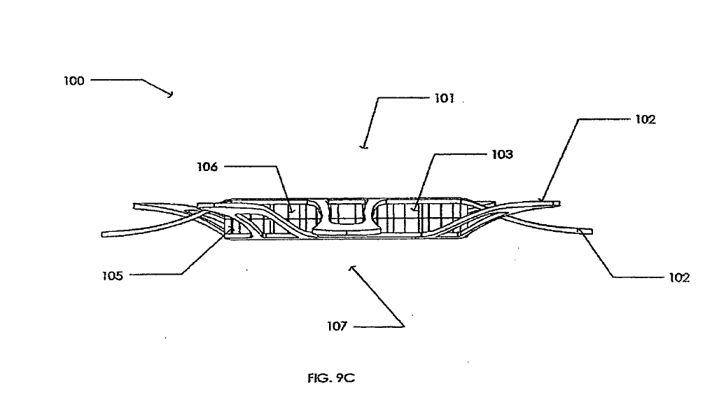

FIGS. 9A-9C illustrate an embodiment according to the invention that is similar to the embodiment described in relation to FIGS. 2A-2B above. FIGS. 9A-9C illustrate in more detail from a perspective view from first end 101 of an embodiment according to the invention in its deployed configuration. Biological pacemaker 100 comprises anchors 102 disposed at both first end 101 and second end 107 for securing biological pacemaker 100 on opposite sides of a septal wall of a subject. Biological pacemaker 100 also comprises chamber 105, which houses a population of cells 104 for the expression of a pacemaker gene and conduction of electrical current from the cells in the chamber to the cardiac myocytes of the patient. Sides 103 of chamber 105 comprise porous membrane 106, and first end 101 and second end 107 comprise porous membrane 108. Porous membranes 106 and 108 comprise pores having functions similar to those described in relation to FIG. 1. When biological pacemaker 100 is implanted in a patient, first end 101 and second end 107 interface most with the blood of the right and left atria respectively (see FIG. 1). Accordingly, membrane 108 may comprise a different porosity than membrane 106, in order to, for example, maximize entry of oxygen or achieve another objective of chamber 105 related to interface with the blood of a subject. Similarly, the porosity of membrane 106 of side 103 may be specifically designed to, for example, maximize the conduction of electrical current from the interior of chamber 105 to the septal wall of the subject.

Further, sides 103 may comprise peaks 108 and valleys 111, thereby increasing the surface area of sides 103, thereby increasing the exposure of porous membrane 106 to the blood of the subject and the septal wall of the subject. Increasing the exposure of membrane 106 may enhance the function of membrane 106 and both the interface of cells 104 with the blood of the subject and the interface of cells 106 with the endogenous cells of the subject. Electrical conductivity between cells 104 and the endogenous cells of the subject may thereby be enhanced, as well as nutrient and waste transfer of cells 104.

Similar to the embodiment discussed in relation to the embodiments described above, biological pacemaker 100 and anchors 102 may be reversibly deployable, allowing removal of biological pacemaker 100 from a subject in a minimally invasive manner. Further, chamber 105 is removable from biological pacemaker 100, allowing refilling or replacement of chamber 105.

FIG. 9C illustrates a side view of the embodiment of FIG. 9A in its deployed configuration. When implanted in a subject, sides 103 abut the interior of the aperture of the septal wall of the subject. Anchors 102 secure biological pacemaker 100 within the aperture of the septal wall of the subject, against opposite sides of the septal wall.

Analogous devices to induce or enhance muscle contraction in areas other than the heart are possible for the treatment of for example, obesity, stress incontinence, and other disorders. Such devices may be used in relation to stomach, esophageal, uterine, ureteral, urethral, bladder, jejunum or ileum smooth muscle cells.

While particular forms of the invention have been illustrated and described above, the foregoing descriptions are intended as examples, and to one skilled in the art it will be apparent that various modifications can be made without departing from the spirit and scope of the invention.

1. An implantable chamber for the delivery and maintenance of cells for the treatment of a cardiac condition, said chamber comprising one or more walls defining a substantially hollow interior, said one or more walls comprising one or more pores, said one or more pores configured to allow the passage of molecules related to the cells' respiration, said one or more pores configured to prevent the passage of the cells therefrom.

2. The chamber according to claim 1 further comprising one or more anchors.

3. The chamber according to claim 1 further comprising a delivery configuration and a deployed configuration.

4. The chamber according to claim 1 wherein said cells comprise a pacemaker gene.

5. The chamber according to claim 1 wherein said one or more pores are generally between 0.1 and 10.0 micrometers in diameter.

6. The chamber according to claim 1 wherein said substantially hollow interior further comprises cells and a synthetic conductive interface with said cells.

7. The chamber according to claim 1 wherein said one or more walls comprises a matrix structure.

8. The chamber according to claim 1 further comprising one or more conductive electrodes extending therefrom.

9. The chamber according to claim 1 further comprising an electrically conductive grid.

10. The chamber according to claim 9 wherein said grid is treated to prevent the overgrowth of endogenous cells.

11. The chamber according to claim 9 wherein said grid is treated to enhance the overgrowth of endogenous cells.

12. The chamber according to claim 1 wherein said one or more pores is configured to prevent the passage of antibodies or endogenous cells therethrough.

13. The chamber according to claim 1 wherein said chamber comprises a surface, wherein said surface is treated to prevent the overgrowth of endogenous cells thereon.

14. The chamber according to claim 1 wherein said chamber comprises a surface, wherein said surface is treated to enhance the overgrowth of endogeneous cells.

15. An implantable chamber for the delivery and maintenance of viable cells for the treatment of a cardiac condition.

16. The chamber according to claim 15 wherein said cardiac condition is a cardiac rhythm disorder.

17. The chamber according to claim 4 wherein said cells comprise stem cells treated to express a pacemaker gene by electroporation, transfer through liposomes, a plasmid, a viral vector, non-viral vector, naked DNA, cationic liposomes, conjugated or mixed vectors, dendrimers, cationic polymers, nanohydrogels, crosslinked micelles, cell-penetrating peptides, cell targeting peptides or other suitable method.

18. The chamber according to claim 2 wherein said one or more anchors comprises one or more shape memory materials.

19. The chamber according to claim 1 wherein said one or more walls comprise one or more metals or one or more polymers.

20. The chamber according to claim 1 wherein said one or more walls comprise ePTFE.

21. The chamber according to claim 1 wherein said one or more walls comprise a membrane prepared according to any suitable nanopore membrane technology.

22. The chamber according to claim 1 wherein when implanted in a subject, said cells are capable of conducting electrical current to the endogenous cells of the subject.

23. The chamber according to claim 1 wherein when said chamber is implanted in the heart of a subject, said chamber allows conductivity of electrical impulses from the cells within the chamber to the endogenous cardiac myocytes of the subject.

24. The chamber according to claim 1 wherein said chamber is percutaneously implantable in the atrial septal wall of a subject.

25. The chamber according to claim 15 wherein said chamber is percutaneously implantable in the atrial septal wall of a subject.

26. An artificial sinoatrial node comprising an implantable chamber comprising viable cells expressing a pacemaker gene.

27. An artificial atrioventricular node comprising an implantable chamber comprising viable cells expressing a pacemaker gene.

28. A method for the minimally invasive treatment of a cardiac condition comprising the steps of:
providing a chamber comprising viable cells, said chamber comprising a delivery configuration and a deployed configuration;
accessing the right atrium or the right ventricle of a subject;
creating an aperture in the atrial or ventricular septal wall;
delivering the chamber to the aperture in the atrial or ventricular septal wall; and
deploying the chamber within the aperture in the atrial or ventricular septal wall.

29. The method according to claim 28 wherein said chamber comprises one or more anchors, with the added step of deploying the one or more anchors for securing the chamber within the atrial or ventricular septal wall.

30. The method according to claim 29 wherein said cells comprise a pacemaker gene.

31. The method according to claim 28 wherein said step of accessing the right atrium comprises the steps of accessing the femoral vein and the inferior vena cava with a catheter.

32. The method according to claim 28 wherein said chamber is configured to maintain said viable cells following delivery of said chamber.

33. The method according to claim 28 wherein said chamber is configured to allow electrical conductivity from the interior of said chamber to the endogenous cells of a subject.

34. The method according to claim 28 wherein said cardiac condition is a cardiac rhythm disorder.

35. The method according to claim 28 wherein said chamber comprises one or more walls comprising one or more pores, said one or more pores configured to allow the passage of molecules related to the cells' respiration, said one or more pores configured to prevent the passage of the cells therefrom.

36. The method according to claim 28 wherein said chamber comprises one or more walls, wherein said one or more walls comprise ePTFE.

37. The method according to claim 28 wherein said chamber comprises one or more walls, wherein said one or more walls comprise a membrane prepared according to any suitable nanopore membrane technology.

38. The chamber according to claim 1 wherein said one or more walls comprise an exterior membrane comprising one or more projections thereby increasing the surface area of the membrane.

39. The chamber according to claim 15 wherein said one or more walls comprise an exterior membrane comprising one or more projections thereby increasing the surface area of the membrane.

40. The chamber according to claim 1 wherein said chamber comprises one or more releasable anchors.

41. The chamber according to claim 3 wherein said deployed configuration is substantially reversible.

42. The chamber according to claim 40 wherein said chamber is readily exchangeable.

43. The chamber according to claim 41 wherein said chamber is readily exchangeable.

44. The method according to claim 28 wherein said chamber is reversibly deployable, with the additional step of removing said chamber from the atrial or ventricular septal wall.

45. The chamber according to claim 6 wherein said chamber further comprises one or more conductive fibers in communication with said cells and the exterior of the chamber.

46. The chamber according to claim 45 further comprising an electrically conductive grid, wherein said one or more conductive fibers is in communication between said cells and said grid.

47. The chamber according to claim 6 wherein said synthetic conductive interface comprises a plurality of sintered spherical structures.

48. The chamber according to claim 47 wherein said one or more walls comprise a microporous membrane extending over the exterior of said sintered spherical structures.

49. The chamber according to claim 1 wherein said chamber is percutaneously implantable in the ventricular septal wall of a subject.

50. The chamber according to claim 15 wherein said chamber is percutaneously implantable in the ventricular septal wall of a subject.

51. The method according to claim 28 wherein the step of accessing the right ventricle comprises the steps of accessing the right atrium, passing through the tricuspid valve, and entering the right ventricle.

52. The method according to claim 35 wherein said pores are configured to allow the passage of neurological and hormonal molecules which drive the sympathetic and parasympathetic systems.

53. The method according to claim 35 wherein said pores are configured to prevent the passage of antibodies or endogenous cells.

54. The chamber according to claim 1 wherein said pores are configured to allow the passage of neurological and hormonal molecules.

55. The chamber according to claim 38 wherein said one or more projections increases the structural strength of the membrane.

56. The chamber according to claim 39 wherein said one or more projections increases the structural strength of the membrane.

57. An implantable chamber comprising cells for promotion of and maintenance of sustained electrical conductivity between living cells.

58. An implantable chamber for promotion of and maintenance of sustained electrical conductivity between implanted cells and endogenous cells via one or more synthetic conductive interfaces.

59. An implantable chamber for promotion of and maintenance of sustained electrical conductivity between implanted cells and endogenous cells via synthetic depolarization channels.

60. An implantable chamber for implanting viable, immunoisolated, electrically conductive cells into a subject for treatment to induce or enhance muscle contraction.

61. The chamber according to claim 9 wherein said grid is treated to promote neovascularization.

62. The chamber according to claim 9 wherein said grid is treated to enhance the overgrowth of endogenous cells and to promote neovascularization.

63. The chamber according to claim 1 wherein said chamber comprises a surface, wherein said surface is treated to promote neovascularization.

64. The chamber according to claim 1 wherein said chamber comprises a surface, wherein said surface is treated to enhance the overgrowth of endogeneous cells and to promote neovascularization.
